(19) United States
(12) Patent Application Publication
(10) Pub. No.: US 2006/0223891 A1
(43) Pub. Date: Oct. 5, 2006

(54) Use of alverine, alone or in combination with a tricyclic antidepressant or an antidepressant which is a specific inhibitor of serotonin reuptake for treatment of depression

(75) Inventors: Jacques Migeon, Seattle, WA (US); Frédéric Revah, Paris (FR)

Correspondence Address: FOLEY AND LARDNER LLP;SUITE 500, 3000 K STREET NW, WASHINGTON, DC, 20007, US

(73) Assignee: CEREP

(21) Appl. No.: 10/560,449

(22) PCT Filed: Jun. 11, 2004
(86) PCT No.: PCT/FR04/01462

§ 371 Date: Mar. 21, 2006

(30) Foreign Application Priority Data
Jun. 13, 2003 (FR) 0307176
Apr. 30, 2004 (FR) 0404639

(51) Int. Cl. A61K 31/137

(52) U.S. Cl. 514/649

(57) ABSTRACT

The present invention relates to the utilisation of Alverine or its metabolites, alone or in combination with a tricyclic antidepressant or a specific inhibitor antidepressant of serotonin recapture, for the preparation of pharmaceutical compositions for the treatment of depression.

Depression is one of the most frequently occurring psychological disorders. In France, the rate of depressives is 14.9%, whereof close to a third is not receiving any medical treatment. The prevalence of declared depression has been multiplied by 6 since 1970. The risk of presenting serious depression throughout a lifetime varies, according to studies, from 10 to 25% for women and from 5 to 12% for men.

The depressive syndrome is associated with mood swings (feelings of sadness, abandonment, humiliation, devaluing), psychomotor inhibition (fatigue, daily powerlessness, difficulty in concentration), manifest anxiety (often in the foreground) with quasi-constant somatic difficulties (oppression, spasms, disturbed sleep, loss of appetite, sexual dysfunction).

The discovery of antidepressants at the end of the fifties marked a veritable therapeutic revolution in the world of neuropsychiatry. Antidepressives are capable, over a period of two to three weeks, of improving a depressive mood and supporting moral suffering. If the first indication of antidepressants is evidently endogenous unipolar depression, it is necessary to know the indication extensions which now concern other psychiatric entities such as depressive episodes of bipolar psychoses, certain states of anxiety, obsessive compulsive disorders, behavioural disorders, eating disorders but also other nosographic contexts such as therapeutic treatment of certain pains.

Tricyclic antidepressants (TCA) with amitriptyline (Laroxyl®) and imipramine (Tofranil®) were the first to be discovered, followed by inhibitors of monoamine oxydase (IMAO), irreversible and non-selective, such as phenelzine (hydrazine), pargyline (class of acetylenics) and iproniazude (Marsilid). Undesirable effects, in particular orthostatic hypotension, dryness in the mouth, drowsiness, constipation, adaptation disorders, but also a proconvuIsivant effect and cardiotoxicity of TCA (especially in the event of overdose) and hypertensive crises of IMAO (interactions with alimentary tyrarnine, as well as numerous medicinal interactions) have shunted research towards novel molecules of identical therapeutic efficacy, but having better acceptability.

The notion of specificity then appeared with specific inhibitors of serotonin (5-hydroxytryptamine or 5HT). Clinical trials of phase III have demonstrated for these -novel -molecules an efficacy equivalent to first-generation antidepressants and greater tolerance, especially in the event of overdose. However, there are unwanted effects with molecules. Most frequently they concern the digestive tract, with nausea, vomiting and, to a lesser degree, constipation and anorexia. Cases of insomnia are described, as are cephalea, hypersudative access and a sexual dysfunction (low libido, premature ejaculation). Weaning syndromes have been described, giving rise to the rule of posologic decline when treatment is to be discontinued.

The serotoninergic syndrome, often misunderstood, is associated with certain overdoses or interactions and justifies an immediate halt to treatment. It can cause hospitalisation, and in exceptional circumstances the involvement of vital prognosis. It links a set of symptoms of digestive order (diarrhoea), vegetative: (sweating, thermal deregulation, hypo- or hypertension), motor (myoclonia, trembling), neuropsychic (confusion, agitation, even coma).

The discovery of the 2 forms A, and B of monoamine oxydase, differing from one another by the affinity of form A for NA and 5HT and of form B for dopamine (DA), has lead to selective and reversible inhibitors of monoamine oxydase A or B. The interest in selective inhibition A or B is to let one of the activities A, or B, persist, sufficient for destroying tyramine which, in patients treated by non-selective IMAO, was at the origin of numerous unwanted effects such as hypertensive access.

In this way, moclobemide (Moclamone®), befloxatone and toloxatone (Humoryl®) are distinguished as selective and reversible inhibitors of monoamine oxydase A. There is, however, the risk of inducing serotoninergic syndromes, above all when their prescription succeeds that of an ISRS. (specific inhibitor of serotonin recapture).

For recent antidepressants now on the market, their therapeutic effect results from simultaneous inhibition of the recapture of serotonin (5HT) and noradrenaline (NA) and they accumulate the resulting secondary effects. Thus, mirtazapine (Norset®), milnacipran (Ixel®) and venlafaxine (Effexor®) act at the same time on noradrenergic tracts and on serotoninergic tracts. Yet, they are no longer devoid of unwanted effects, since mirtazapine frequently causes significant weight gain. Milnacipran (Ixel®) and venlafaxine (Effexor®) cause an elevation in diastolic arterial pressure as well as nervousness and anorexia.

Therefore, pharmacopoeia offers efficacious antidepressant products, though not devoid of secondary effects. The current problem being faced is the existence of efficacious treatment for depression, which comprises the fewest unwanted effects possible, and zero or virtually toxicity.

One of the aims of the present invention is to propose products allowing treatment of depression, but to a large degree lacking in the abovementioned secondary effects.

Alverine is a medication classically used as antispasmodic for treatment of functional abdominal manifestations especially with meteorism. The present invention is based on the unexpected prominence given to the antidepressive properties of Alverine,

The mode of action of Alverine is different to that of tricyclic antidepressants and to that of specific or non-specific inhibitors of the recapture of serotonin, since Alverine interacts marginally with serotonin or noradrenaline recapture systems.

The advantage of Alverine is that this product, commercially available now for over 50 years, has a very low toxicity and secondary effects which are highly limited over more than half a century, as compared to the classic antidepressants described hereinabove.

The present invention describes the anti-depressive properties of Alverine in animals.

The object of the present invention is thus utilisation of Alverine or its metabolites, as well as esters and pharmaceutically acceptable salts for the preparation of pharmaceutical compositions for treating depression.

Alverine is understood to mean N-ethyl-3,3′-diphenyldipropylamine

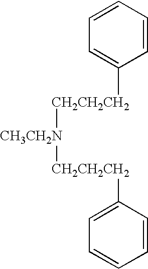

Alverine metabolites are understood to mean inter alia mono- or polyhydroxylated derivatives on phenyl nuclei and mono- or polyhydroxylated or mono- or polycarboxylated derivatives on aliphatic chains. Three of the principal metabolites identified by way of example after incubation of Alverine with microsomes of human liver are:

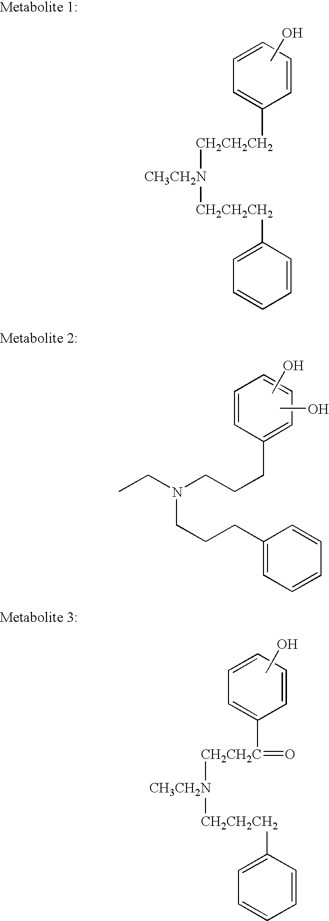

Pharmaceutically acceptable salts are understood to mean salts of addition of Alverine, which can be obtained by reaction of this compound with a mineral acid or organic solvent according to a method known per se. Examples of acids which can be used to this effect are the following: hydrochloric, bromhydric, sulfonic, phosphoric, sulfonic 4-toluoene, suIfonic methane, sulfonic cyclohexyl, oxalic, succinic, formic, fumaric, maleic, citric, aspartic, cinnamic, lactic, glutamic, N-acetylaspartic, N-acetylglutamic, ascorbic, malic, benzoic, nicotinic and acetic, while Alverine citrate and tartate have been used widely in spasmolytic pharmaceutical preparations.

Examples of esters on the hydroxy function are carboxylic acid esters having from 1 to 6 carbon atoms.

Even though Alverine is known for its antispasmodic activity and is utilised in the treatment of functional abdominal manifestations, especially with meteorism, its action as antidepressant agent has never been described or suggested.

Alverine, its metabolites, its salts, and especially the citrate and the esters can be administered in a pharmaceutically acceptable form via one of the different ways known for this type active ingredient.

Preferably, the object of the invention is the utilisation of Alverine or its metabolites in which the pharmaceutical composition is administered orally, sublingually, buccally, sub-cutaneously, transdermally, locally, rectally, intranasally, or injectabIy, in particular intraperitoneally, intravenously or intramuscularly.

Preferably, the object of the invention is the utilisation of Alverine or its metabolites for the preparation of a pharmaceutical composition, which can be administered orally, especially in the form of capsules or tablets.

The active substances in the pharmaceutical compositions according to the present invention can be in any of the usual oral galenic forms comprising tablets, capsules and liquid preparations such as elixirs and suspensions containing diverse masking substances of dyes, flavour and stabilisation.

To produce the oral galenic forms according to the present invention, especially capsules, the active substance can be mixed in with various conventional materials such as starch, calcium carbonate, lactose, sucrose and dicalcic phosphate to facilitate the process of encapsulation. Magnesium stearate, as additive, provides a useful function as lubricant, if necessary.

In certain cases it can be interesting to provide forms with controlled release and especially prolonged release via known galenic forms.

Similarly, the object of the invention is the utilisation of Alverine or its metabolites for the preparation of a pharmaceutical composition, which can be administered injectably.

The active substances of the pharmaceutical compositions according to the present invention can be dissolved or placed in suspension in a pharmaceutically acceptable sterile injectable liquid, such as sterile water, a sterile organic solvent or a mixture of these two liquids for intravenous administration. Other ways of administration can comprise, though are not limited to, sub-cutaneous implants, as well as buccal, sublingual, transdermic, topical, intranasal or rectal administrations. Biodegradable and non-biodegradable administration systems can also be employed here.

According to a particular embodiment, the object of the invention is the utilisation of Alverine or its metabolites, salts or esters for the preparation of a pharmaceutical. composition administrable according to one of the preceding ways in a dose from 1 to 1000 mg of active ingredient for a composition formulated in the form of capsules or tablets, or from 4.1 to 500 mg of active ingredient for a composition formulated in the form of suppositories, pomades, creams, gels or aerosol preparations, administered in human therapy in one or more daily doses for an adult of an average weight of 60 to 70 kg.

Within the scope of use for animals, the daily dose is between 0.01 and 100 mg per kg.

Alverine, or its metabolites, salts or esters can also be used according to the object of the present invention in combination with a tricyclic antidepressant compound. Preferably, the tricyclic antidepressant compound is imipramine. Alverine, or its metabolites, salts or esters can likewise be utilised according to the object of the present invention in combination with a specific inhibitor antidepressant compound of serotonin recapture.

Also preferably, the specific inhibitor antidepressant compound of serotonin recapture is fiuoxetine.

Within the scope of the present invention, it is possible to provide administration, of mixtures of the preceding compounds, but in the majority of cases, considering the requisites of health authorities, administration will be done in the form of coprescription. The products could be administered simultaneously or separately over time in consideration of their particularities and especially of their bioavailability.

The ratios of the doses of the different products naturally depend on the products used, but preliminary trials have shown that the 1/1 associations of Alverine and antidepressant would enable the doses administered to be divided by 3 to obtain the same antidepressant effect.

Preferably, ratios of active ingredients by weight of between 1/4 and 4/1 between Alverine and the antidepressant will be used, which should allow the administered doses of each compound to be divided at least by 2.

The compounds according to the present invention are administered simultaneously, separately or staggered over time.

According to a second aspect, the object of the present invention is also a pharmaceutical composition, characterised in that it is a combination product comprising at least the Alverine compound or its metabolites, salts or esters and at least one tricyclic antidepressant compound for simultaneous use, separately or staggered over time for treating depression.

Preferably, the pharmaceutical composition according to the present invention is characterised in that it comprises ratios of doses by weight of Alverine and tricyclic antidepressant of between 1/10 and 10/1. More preferably, the ratios of doses by weight are between 1/4 and 4/1.

The tricyclic antidepressant compound is preferably imipramine.

Other tricyclic antidepressants can be used, especially clomipramine, amitriptyline, maprotiline, amoxapine, desipramine, nortriptyline, demexiptaine, dibenzepine, dosulepine, doxepine, metapramine, noxiptiline, opipramol, propizepine, quinuprainine, and trimipramine.

According to a third aspect, another object of the present invention is a pharmaceutical composition, characterised in that it is a combination product comprising at least the Alverine compound or its metabolites, salts or esters and at least a specific inhibitor antidepressant compound of serotonin recapture for simultaneous use, separately or staggered over time for treating depression.

Preferably, the pharmaceutical composition according to the present invention is characterised in that it comprises ratios of doses by weight of Alverine and specific antidepressant inhibitor of serotonin recapture of between 1/10 and 10/1. More preferably, the ratios of doses by weight are between 1/4 and 4/1.

Preferably, the. specific antidepressant inhibitor compound of serotonin recapture is fluoxetine.

Other inhibitors of serotonin recapture can be utilised, especially paroxetine, citaIorpam, fluvoxamine, sertraline.

Treatment of depression is understood to mean treatment of all the phenomena of depressive type, as well as the treatment of unique. depressive episodes and recurrent depressive episodes or major depressions, but also the treatment of depressive episodes. of bipolar or cyclothymic disorders, and apparent disorders.

The present invention also relates to a method of treating depression comprising administration of a composition according to the present invention to a patient having need of such treatment.

Said composition comprises Alverine or its metabolites, alone or in combination with a tricyclic antidepressant or a specific inhibitor antidepressant of serotonin recapture.

In the case of a combination of Alverine and a tricyclic antidepressant or a specific inhibitor antidepressant of serotonin recapture, the ratios of doses by weight are from 1/10 to 10/1 and preferably from 1/4 to 4/1.

The processes for preparing Alverine from phenylpropyl chloride and ethylaznine, in an alkaline medium are described in Külz et al., Report 72,2165 (1939) and its galenic is also known.

The mechanism for synthesising metabolites 1, 2 and 3 of Alverine are illustrated by diagrams 1, 2 and 3. The experimental protocols for the synthesis of metabolites 1 para-OH and ortho-OH are described in the patent W092/02488 by W.J. Horgan and illustrated by diagram 1. Diagrams 1, 2 and 3 are presented hereinbelow:

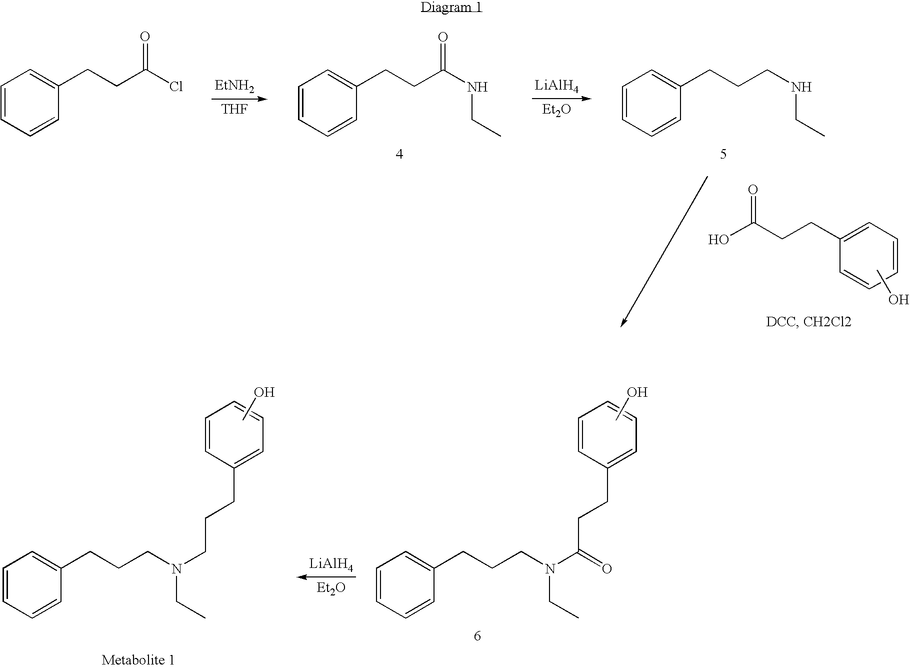

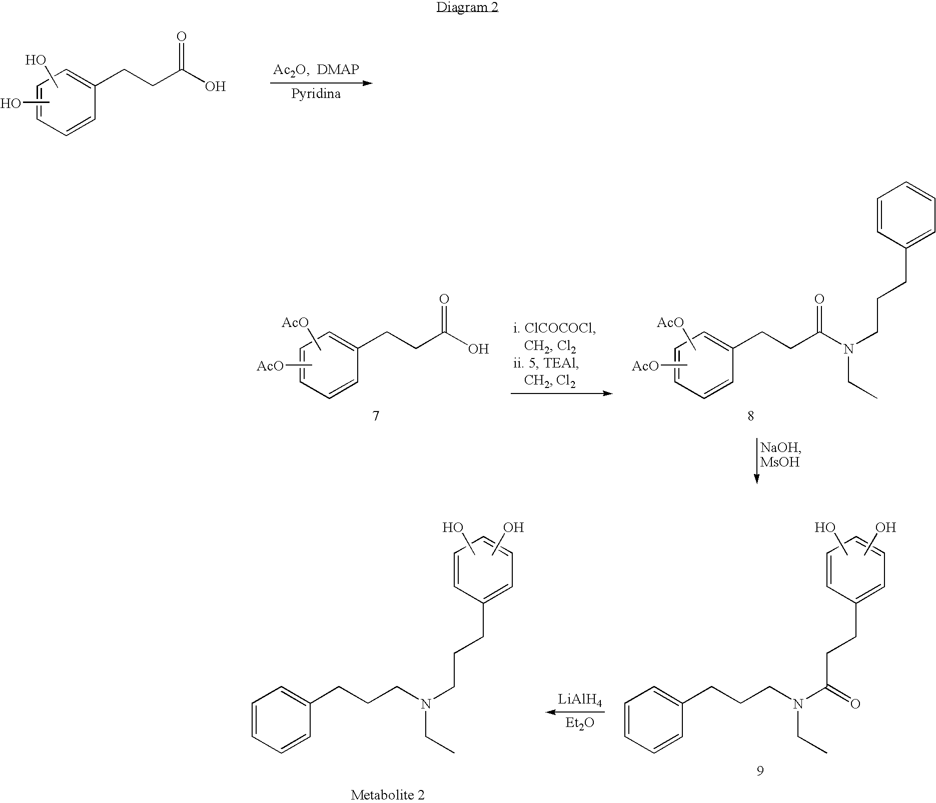

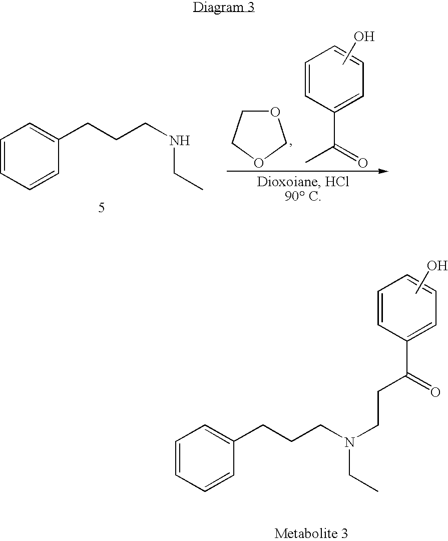

The present invention will be better understood by means of the following description, which refers to examples of antidepressive activity tests on Alverine, alone or in combination with other antidepressants, administered to mice according to the present invention.

It goes without saying all the same that these examples are given purely by way of illustration of the object of the invention, whereof they would in no way be construed as a limitation.

### FIGURES

FIG. 1 is a presentation histogram of the results obtained by an antidepressive activity test on Alverine administered intraperitoneally on a batch of mice, presented in Table 1 and described in example 1.

FIG. 2 is a presentation histogram of the results obtained by an antidepressive activity test on Alverine administered orally on a batch of mice, presented in Table 2 and described in Example 2.

FIG. 3 is a presentation histogram of the results obtained by an antidepressive activity test on Alverine and imipramine administered intraperitoneally on a batch of mice, presented in Table 3 and described in Example 3.

FIG. 4 is a presentation histogram of the results obtained by an antidepressive activity test on Alverine and fluoxetine administered intraperitoneally on a batch of mice, presented in Table 4 and described in Example 3.

### EXAMPLES

The examples given hereinbelow illustrate the invention without limiting it in any way:

### Example 1

Antidepressive Activity Test on Alverine administered intraperitoneally on a batch of mice.

To establish the advantages according to the present invention a study was carried out on a batch of 50 mice. They were divided into 5 groups of 10 mice each. These are Swiss mice CD1 (CD-1®(ICR) IGS (Charles River France) weighing between 25 and 35 g.

They were placed in a room at a temperature of between 19.5 and 24.5° C. and a relative humidity of 45 to 65% with a light/dark cycle of 12 h, ad libitum access to filtered water and pellets of laboratory-standard food.

They are placed 15 to 20 per cage, over an acclimatising period of at least 5 days prior to the tests. They are identified by marking on the fur.

The substance to be tested is Alverine citrate (Sigma, in the form of dry powder, with a salt/base ratio of 1.68) comparatively to imipramine chlorhydrate (Sigma, in the form of dry powder, with a salt/base ratio of 1.13).

The first group is the specimen group: it is treated only by excipient.

The second group is treated with Alverine at a dose of 3 mg/kg

The third group is treated with Alverine at a dose of 10 mg/kg

The fourth group is treated with Alverine at a dose of 30 mg/kg

The fifth group is treated with imipramine (tricyclic antidepressant) at a dose of 10 mg/kg

The doses are expressed in terms of free active substances. The substances are prepared extemporaneously in the excipient. The treatments are administered 30 minutes prior to the test in a coded and random order intraperitoneally with a volume of 10 ml/kg.

Thirty minutes following administration the five groups of mice are subjected to the forced swimming test, in a vertical Plexiglas cylinder (height 24 cm, diameter 9 cm) containing water (height 6 cm, temperature 18-22° C.). The total duration of immobility is measured over the last four minutes of the test, six minutes in total. A mouse is deemed immobile when it ceases struggling and floats in the water without movements superfluous to those allowing it to keep its head above water. A drop in immobility time is the reflection of an antidepressant effect.

The forced swim test is a pre-clinical behavioural model, which has good predictive validity and is widely employed for determining the efficacy of antidepressant medications (Borsini and Meli, 1988).

The results are expressed in total duration of immobility in seconds and as a percentage of variation of the total duration of immobility calculated from the average value of the sample group.

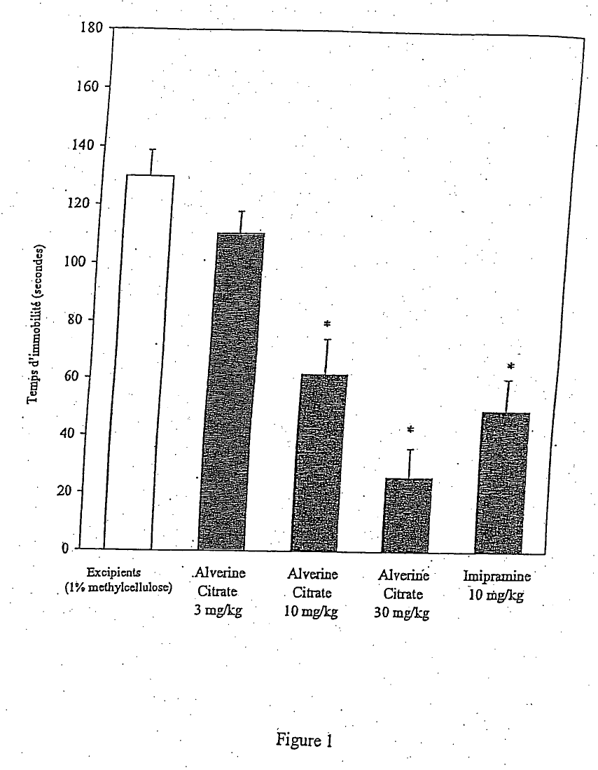

The statistical significance between the treated groups and the sample group is determined by a Dunnett test using the residual variation according to analysis of the variance (P<0.05). The data are analysed using <<SigmaStat>> software. The results obtained are presented in the form of the following table, and in the form of a histogram, in FIG. 1.

TABLE 1
results obtained by an antidepressive
activity test of Alverine administered
intraperitoneally on a batch of mice.

| | Excipient (1% | Alverine | Alverine | Alverine | |
|---|---|---|---|---|---|
| Substances | methyl | citrate | citrate | citrate | Imipramine |
| Doses mg/kg | cellulose) | 3 mg/kg | 10 mg/kg | 30 mg/kg | 10 mg/kg |
| immobility | 97 | 118 | 3 | 12 | 65 |
| time (sec) | 107 | 128 | 29 | 97 | 67 |
| | 144 | 82 | 86 | 3 | 70 |
| | 171 | 151 | 28 | 66 | 1 |
| | 144 | 132 | 30 | 36 | 89 |
| | 136 | 127 | 90 | 0 | 3 |
| | 79 | 88 | 99 | 15 | 9 |
| | 128 | 85 | 65 | 7 | 38 |
| | 132 | 99 | 129 | 16 | 99 |
| | 160 | 93 | 57 | 7 | 53 |
| Average error type | 129.8 | 110.3 | 61.6 | 25.9 | 49.4 |
| on | 9.0 | 7.6 | 12.5 | 10.0 | 11.2 |
| average | | | | | |
| Dunnett test | P < 0.05 | ns | * | * | * |
| % of | | −15 | −53 | −80 | −62 |
| variation | | | | | |

Administration is 30 minutes prior to the test.
N = 10 animals per group
* indicates a significant difference for p < 0.05 (Dunnett test)
ns indicates an insignificant result

It is observed that the more significant the Alverine dose administered, the more the immobility time of the mice diminishes, indicating an antidepressant effect proportional to the dose (FIG. 1).

In addition, it is observed that the mice of the third group treated at 10 mg/kg Alverine exhibit an immobility time comparable to that of the mice of the fifth group treated at 10 mg/kg of imipramine.

It can thus be concluded that Alverine, injected intraperitoneally has a significant antidepressant effect in mice and just as important as Imipramine, in comparable doses.

### Example 2

Antidepressive Activity Test on Alverine administered orally on a batch of mice.

To establish the advantages according to the present invention a study, was carried out on a batch of 50 mice. They were divided into 5 groups of 10 mice each. These are Swiss mice CD1 (CD-1® (ICR) IGS (Charles River France) weighing between 25 and 35 g.

They were placed in a room at a temperature of between 19.5 and 24.5° C. and a relative humidity of 45 to 65% with a light/dark cycle of 12 h, ad libitum access to filtered water and pellets of laboratory-standard food.

They are placed 15 to 20 per cage, over an acclimatising period of at least 5 days prior to the tests. They are identified by marking on the fur.

The substance to be tested is Alverine citrate (Sigma, in the form of dry powder, with a salt/base ratio of 1.68) comparatively to imipramine chlorhydrate (Sigma, in the form of dry powder, with a salt/base ratio of 1.13).

The first group is the specimen group: it is treated only by excipient.

The second group is treated with Alverine at a dose of 10 mg/kg

The third group is treated with Alverine at a dose of 30 mg/kg

The fourth group is treated with Alverine at a dose of 100 mg/kg

The fifth group is treated with imipramine (tricyclic antidepressant) at a dose of 30 mg/kg The doses are expressed in terms of free active substances. The substances are prepared extemporaneously in the excipient. The treatments are administered 1 hour prior to the test in a coded and random order intraperitoneally with a volume of 10 ml/kg.

One hour following administration the five groups of mice are subjected to the forced swimming test, in a vertical Plexiglas cylinder (height 24 cm, diameter 9 cm) containing water (height 6 cm, temperature 18-22° C.). The total duration of immobility is measured over the last four minutes of the test, six minutes in total. A mouse is deemed immobile when it ceases struggling and floats in the water without movements superfluous to those allowing it to keep its head above water. A drop in immobility time is the reflection of an antidepressant effect.

The forced swim test is a pre-clinical behavioural model, which has good predictive validity and is widely employed for determining the efficacy of antidepressant medications (Borsini and Meli, 1988).

The results are expressed in total duration of immobility in seconds and as a percentage of variation of the total duration of immobility calculated from the average value of the sample group.

The statistical significance between the treated groups and the sample group is determined by a Dunnett test using the residual variation according to analysis of the. variance (P<0.05). The data are analysed using <<SigmaStat>> software.

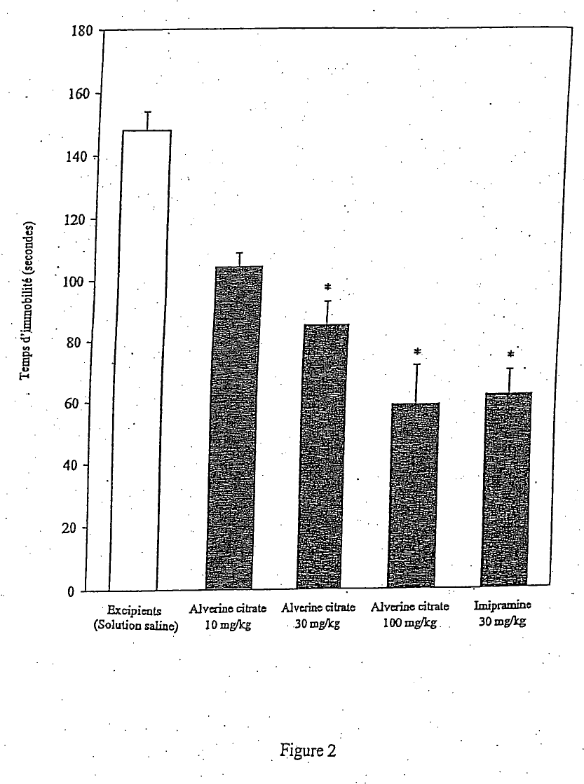

The results obtained are presented in the form of the following table, and in the form of a histogram, in FIG. 2.

TABLE 2
results obtained by an antidepressive activity
test of Alverine administered orally on a batch of mice.

| | | Alverine | Alverine | Alverine | |
|---|---|---|---|---|---|
| Substances | Excipient | citrate | citrate | citrate | Imipramine |
| Doses mg/kg | (saline solution) | 10 mg/kg | 30 mg/kg | 100 mg/kg | 30 mg/kg |
| immobility | 167 | 113 | 113 | 32 | 96 |
| time (sec) | 128 | 86 | 104 | 52 | 52 |
| | 123 | 95 | 80 | 129 | 64 |
| | 126 | 104 | 64 | 67 | 55 |
| | 139 | 111 | 70 | 6 | 5 |
| | 159 | 126 | 105 | 105 | 75 |
| | 163 | 108 | 105 | 75 | 67 |
| | 147 | 78 | 105 | 81 | 70 |
| | 149 | 115 | 41 | 5 | 89 |
| | 179 | 106 | 63 | 37 | 45 |
| Average error type | 148.0 | 104.2 | 85.0 | 58.9 | 61.9 |
| on | 6.0 | 4.5 | 7.8 | 12.8 | 8.1 |
| average | | | | | |
| Dunnett test | P < 0.05 | ns | * | * | * |
| % of variation | | −30 | −43 | −60 | −58 |

Administration is 60 minutes prior to the test.
N = 10 animals per group
* indicates a significant difference for p < 0.05 (Dunnett test)
ns indicates an insignificant result

It is observed that the more significant the Alverine does administered, the more the immobility time of the mice diminishes, indicating an antidepressant effect proportional to the dose (FIG. 2).

In addition, it is observed that the mice of the fifth group treated at 30 mg/kg Imipramine exhibit ah immobility time less than that of the mice of the third group treated at 30 mg/kg of Alverine, but comparable to the mice of the fourth group treated at 100 mg/kg of Alverine.

In addition, no secondary effect was observed in the mice treated orally with Alverine, using the above doses.

It can thus be concluded that Alverine, injected orally has a significant antidepressant effect in mice, even though this effect is comparable to that of Imipramine only in larger doses, and also without generating secondary effects.

### Example 3

Antidepressive Activity Test on Alverine associated with imipramine or fluoxetine administered intraperitoneally on a-batch of mice.

To establish the advantages of a composition comprising Alverine and imipramine or Alverine and fluoxetine a study was carried out on a batch of 120 mice. These are Swiss mice CD1 (CD-1® (ICR) IGS (Charles River France) weighing between 25-and 35 g.

They were placed in a room at a temperature of between 19.5 and 24.5° C. and a relative humidity of 45 to 65% with a light/dark cycle of 12 h, ad libitum access to filtered water and pellets of laboratory-standard food.

They are placed 15 to 20 per cage, over an acclimatising period of at least 5 days prior to the tests. They are identified by marking on the fur.

The substances to be tested are Alverine citrate (Sigma, in the form of dry powder, with a salt/base ratio of 1.68) imipramine chlorhydrate (Sigma, in the form of dry powder, with a salt/base ratio of 1.13) and fluoxetine chlorhydrate (Sigma, in the form of dry powder, with a salt/base ratio of 1.12).

The mice were divided into two test comprising six groups of 10 mice each.

For the first test:

The first group is the specimen group: it is treated only by excipient.

The second group is treated with imipramine at a dose of 3 mg/kg

The third group is treated with Alverine at a dose of 3 mg/kg

The fourth group is treated with Alverine at a dose of 3 mg/kg and imipramine at a dose of 3 mg/kg

The fifth group is treated with imipramine at a dose of 10 mg/kg The sixth group is treated with Alverine at 10 mg/kg

For the second test:

The first group is the specimen group: it is treated only by excipient.

The second group is treated with fluoxetine at a dose of 3 mg/kg

The third group is treated with Alverine at a dose of 3 mg/kg

The fourth group is treated with Alverine at a dose of 3 mg/kg and fluoxetine at a dose of 3 mg/kg

The fifth group is treated with fluoxetine at a dose of 10 mg/kg

The sixth group is treated with Alverine at 10 mg/kg

The doses are expressed in terms of free active substances. The test . substances are prepared extemporaneously in a saline solution. The treatments are co-administered 30 minutes prior to the test in a coded and random order intraperitoneally with a volume of 10 ml/kg (5 ml/kg for each administration).

Thirty minutes following administration the six groups of mice are subjected to the forced swimming test, in a vertical Plexiglas cylinder (height 24 cm, diameter 9 cm) containing water (height 6 cm, temperature 18-22° C.). The total duration of immobility is measured over the last four minutes of the test, six minutes in total. A mouse is deemed immobile when it ceases struggling and floats in the water without movements superfluous to those allowing it to keep its. head above water. A drop in immobility time is the reflection of an antidepressant effect.

The forced swim test is a pre-clinical behavioural model, which has good predictive validity and is widely employed for determining the efficacy of antidepressant medications (Borsini and Meli, 1988).

The results are expressed in total duration of immobility in seconds and as a percentage of variation of the total duration of immobility calculated from the average value of the sample group.

The statistical significance between two treated groups is determined by using a Student test (P<0.05). The data are analysed using <<SigmaStat>> software.

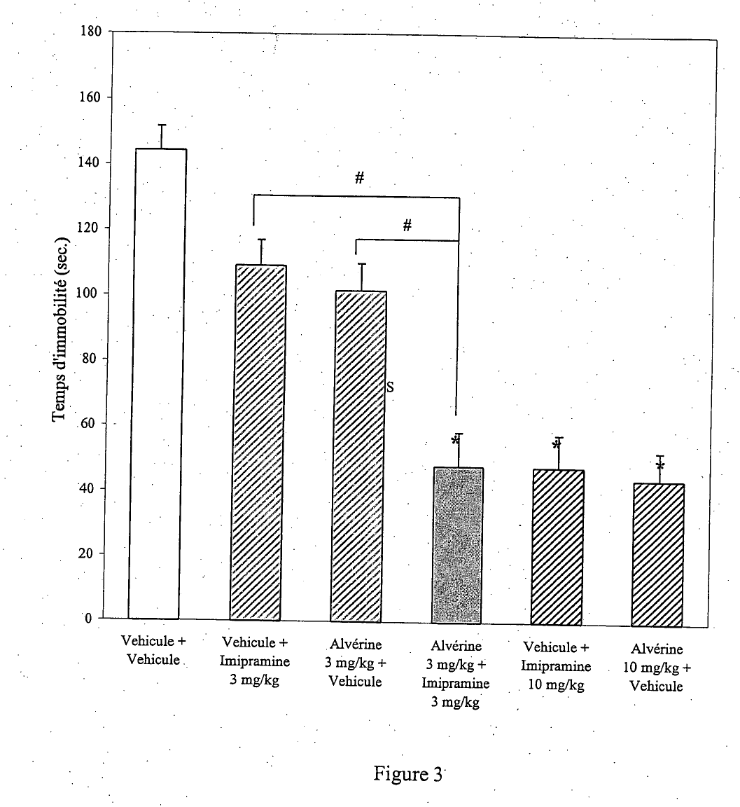

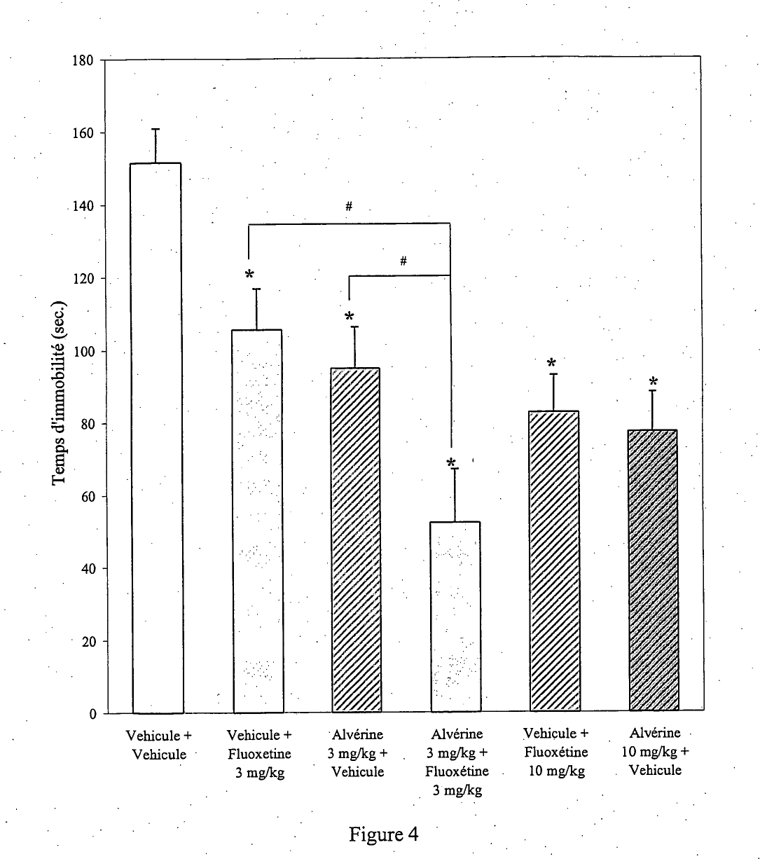

The results obtained are presented in the form of the following table, and in the form of a histogram, in FIGS. 3 and 4.

TABLE 3
results obtained by an antidepressive
activity test of Alverine and imipramine administered
intraperitoneally on a batch of mice.

| Substances | | | | Alverine | | |
|---|---|---|---|---|---|---|
| Doses | | Imipramine | Alverine | 3 mg/kg + imipramine | Imipramine | Alverine |
| mg/kg | Excipient | 3 mg/kg | 3 mg/kg | 3 mg/kg | 10 mg/kg | 10 mg/kg |
| immobility | 125 | 123 | 140 | 77 | 4 | 57 |
| time (sec) | 94 | 121 | 108 | 21 | 0 | 28 |
| | 163 | 121 | 134 | 32 | 62 | 10 |
| | 143 | 70 | 113 | 68 | 38 | 1 |
| | 147 | 74 | 65 | 86 | 54 | 74 |
| | 130 | 122 | 85 | 37 | 45 | 22 |
| | 169 | 93 | 73 | 66 | 92 | 70 |
| | 156 | 94 | 79 | 88 | 26 | 69 |
| | 147 | 141 | 125 | 5 | 77 | 67 |
| | 169 | 133 | 95 | 0 | 80 | 42 |
| Average | 144.3 | 109.2 | 101.7 | 48.0 | 47.8 | 44.0 |
| error type | 7.3 | 7.8 | 8.3 | 10.5 | 9.9 | 8.6 |
| on average | | | | | | |
| Dunnett | P < 0.05 | ns | ns | * | * | * |
| test | | | | | | |
| % of | | −24 | −30 | −67 | −67 | −70 |
| variation | | | | | | |

The compounds to be tested or the vehicle are co-administered intraperitoneally 30 minutes prior to the test (10 ml/kg)
Vehicle: physiological serum
n = 10 animals per group
* indicates a significant difference for p < 0.05 (Dunnett test)
ns indicates an insignificant result
# in FIG. 3 indicates a significant difference for P < 0.05 (Student test).

TABLE 4
results obtained by an antidepressive
activity test of Alverine and fluoxetine administered
intraperitoneally on a batch of mice.

| Substances | | | | Alverine | | |
|---|---|---|---|---|---|---|
| Doses | | Fluoxetine | Alverine | 3 mg/kg + fluoxetine | Fluoxetine | Alverine |
| mg/kg | Excipient | 3 mg/kg | 3 mg/kg | 3 mg/kg | 10 mg/kg | 10 mg/kg |
| immobility | 197 | 125 | 32 | 133 | 122 | 75 |
| time (sec) | 172 | 149 | 119 | 8 | 76 | 63 |
| | 130 | 127 | 138 | 82 | 83 | 75 |
| | 115 | 18 | 117 | 90 | 76 | 77 |
| | 175 | 101 | 110 | 2 | 105 | 46 |
| | 160 | 99 | 73 | 32 | 90 | 6 |
| | 151 | 10 | 117 | 34 | 28 | 113 |
| | 143 | 100 | 47 | 3 | 88 | 89 |
| | 171 | 103 | 124 | 106 | 34 | 104 |
| | 100 | 134 | 72 | 32 | 125 | 125 |
| Average | 151.4 | 105.6 | 94.9 | 52.2 | 82.7 | 77.3 |
| error type | 9.4 | 11.2 | 11.4 | 14.8 | 10.2 | 10.9 |
| on average | | | | | | |
| Dunnett | P < 0.05 | ns | ns | * | * | * |
| test | | | | | | |
| % of | | −30 | −37 | −66 | −45 | −49 |
| variation | | | | | | |

The compounds to be tested or the vehicle are co-administered intraperitoneally 30 minutes prior to the test (10 ml/kg)
Vehicle: physiological serum
n = 10 animals per group
* indicates a significant difference for p < 0.05 (Dunnett test)
ns indicates an insignificant result
# in FIG. 4 indicates a significant difference for P < 0.05 (Student test).

In the test no. 1 (Table 3, FIG. 3), imipramine and Alverine tested alone at 3 mg/kg produce a decrease, statistically insignificant, in the duration of immobilisation as compared to the sample group.

Co-administration of Alverine and imipramine at 3 mg/kg induces a significant antidepressive effect by comparison to the sample group. This effect is significantly greater than the effect produced by each of the compounds alone and is comparable to what is obtained much higher with doses of each compound (10 mg/kg).

In the test no. 2 (Table 4, FIG. 4), fluoxetine and Alverine tested alone at 3 mg/kg produce a decrease, statistically insignificant, in the duratation of immobilisation as compared to the sample group.

Co-administration of Alverine and imipramine at 3 mg/kg induces a significant antidepressive effect by comparison to the sample group. This effect is significantly greater than the effect produced by each of the compounds alone and is comparable to what is obtained much higher with doses of each compound (10 mg/kg).

It can thus be concluded that co-administration of Alverine citrate with imipramine or fluoxetine produces a synergic antidepressant effect in the forced swim test in mice.

In the two associations proposed the doses of each product utilised enables similar results to strongly decrease the administered doses and thus reduce the secondary effect(s) of the compounds used.

1-20. (canceled)

21. A method for treating depression comprising the administration of an effective amount of Alverine 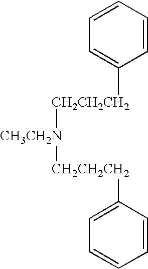
or its metabolites, their salts and their esters, to a subject in need thereof.

22. The method as claimed in claim 21, wherein the metabolites of Alverine are.

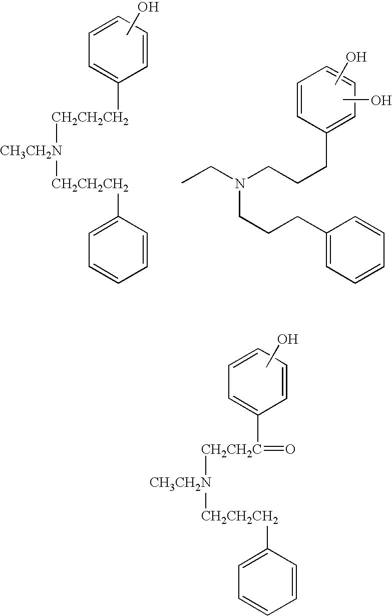

23. The method as claimed in claim 21 wherein said Alverine is administered orally, sublingually, buccally, sub-cutaneously, transdermally, locally, rectally, intranasally, or injectably, in particular intraperitoneally, intravenously or intramuscularly.

24. The method as claimed in claim 23, wherein said Alverine is dosed from 0.1 to 1000 mg.

25. The method as claimed in claim 23 wherein said Alverine is administered in human therapy in one or more daily doses for an adult weighing an average of 60 to 70 kg.

26. The method as claimed in claim 21 wherein said Alverine is administrated in combination with a tricyclic antidepressant compound.

27. The method as claimed in claim 26, wherein said tricyclic antidepressant compound is imipramine.

28. The method as claimed in claim 21 wherein said Alverine is administrated in combination with specific inhibitor antidepressant compound of serotonin recapture.

29. The method as claimed in claim 28, wherein specific inhibitor antidepressant compound of serotonin recapture is fluoxetine.

30. The method as claimed in claim 26, wherein the compounds are administered simultaneously, separately or staggered over time.

31. A pharmaceutical composition, wherein it comprises at least a compound as claimed in claim 21 or its salts or esters and at least a tricyclic antidepressant compound for simultaneous use, separately or staggered over time for treating depression.

32. The pharmaceutical composition as claimed in claim 31, wherein the ratio of dose by weight of Alverine and tricyclic antidepressant is between 1/10 and 10/1.

33. The pharmaceutical composition as claimed in claim 31, wherein the ratio of dose by weight of Alverine and tricyclic antidepressant is between 1/4 and 4/1.

34. The pharmaceutical composition as claimed in claim 31, wherein the tricyclic antidepressant compound is imipramine.

35. A pharmaceutical composition, wherein it comprises at least a compound as claimed in claim 21 or its salts or esters and at least a specific inhibitor antidepressant compound of serotonin recapture for simultaneous utilisation, separately or staggered over time for treating depression.

36. The pharmaceutical composition as claimed in claim 25 wherein the ratio of dose by weight of Alverine and the specific inhibitor antidepressant of serotonin recapture is between 1/10 and 10/1.

37. The pharmaceutical composition as claimed in claim 25, wherein the ratio of dose by weight of Alverine and the specific inhibitor antidepressant of serotonin recapture is between 1/4 and 4/1.

38. The pharmaceutical composition as claimed in claim 25, wherein the specific inhibitor antidepressant compound of serotonin recapture is fluoxetine.

39. A pharmaceutical composition, wherein it comprises at least a compound as claimed in claim 22 or its salts or esters and at least a tricyclic antidepressant compound for simultaneous use, separately or staggered over time for treating depression.

40. A pharmaceutical composition, wherein it comprises at least a compound as claimed in claim 22 or its salts or esters and at least a specific inhibitor antidepressant compound of serotonin recapture for simultaneous utilisation, separately or staggered over time for treating depression.
